(19) United States
(12) Patent Application Publication
(10) Pub. No.: US 2008/0214521 A1
(43) Pub. Date: Sep. 4, 2008

(54) Sensitization of Drug-Resistant Lung Caners to Protein Kinase Inhibitors

(76) Inventor: Martin Schuler, Mainz (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/997,915

(22) PCT Filed: Aug. 7, 2006
(86) PCT No.: PCT/EP2006/065122

§ 371 Date: Feb. 5, 2008

Related U.S. Application Data
(60) Provisional application No. 60/706,701, filed on Aug. 9, 2005.

(51) Int. Cl. A61K 31/553; A61P 35/00

(52) U.S. Cl. 514/211.08

(57) ABSTRACT

The present invention relates to a method of treating non-small cell lung cancer with FLT-3 kinase inhibitor such as PKC412. The invention also relates to a pharmaceutical combination of a FLT-3 kinase inhibitor and an activator of permeabilization of the mitochondrial outer membrane, such as an activator of BAK. It also relates to the use of a pharmaceutical combination of an activator of permeabilization of the mitochondrial outer membrane and a FLT-3 kinase inhibitor for the treatment of non-small cell lung cancer and the use of such a pharmaceutical composition for the manufacture of a medicament for the treatment of same.

### INTRODUCTION

The molecular understanding of cellular signal transduction pathways regulating survival, genetic stability, metabolic activity and proliferation has vastly increased during the past decades. Accordingly, careful analyses conducted in preclinical cancer models and in tumour samples led to the identification of specific deregulations of these pathways as contributing or even causative factors during malignant transformation and cancer progression (1). Against this background, efforts are in place to develop therapies that are tailored for specific targets separating cancer cells from their non-malignant counterparts. The successful clinical application of the small drug kinase inhibitor imatinib in BCR-ABL-positive leukaemias and in gastrointestinal stromal tumours has impressively provided proof-of-principle for such a concept (2). However, pharmacologic inhibitors of apparently less essential signal transduction pathways exhibited only minor clinical activity in unselected patient populations. Further, combining cytotoxic drugs with non-antibody inhibitors so far failed to produce improved clinical outcomes in lung cancer or colorectal cancer (3-6).

Based on these observations we reasoned that cancer cell death induced by pharmacologic kinase inhibitors is actually executed via molecular pathways distinct from those triggered by standard cytotoxic anticancer drugs. Alternatively, both pathways could converge at a common step in signal transduction, which then would constitute a strategic target for breaking drug resistance.

Cytotoxic treatments for patients with advanced non-small cell lung cancer (NSCLC) have only moderate clinical activity. Recently, inhibitors of epidermal growth factor receptor signaling showed efficacy in a subgroup of NSCLC patients, and the modulation of additional signaling pathways holds significant promise. A need exists for cancer therapeutics that target molecular pathways not currently targeted by existing anti-cancer drugs.

### SUMMARY OF THE INVENTION

We studied the induction of apoptosis by the protein kinase C (PKC)-specific inhibitors staurosporine and PKC412 in NSCLC cells. Interestingly, we found that cell lines resistant to cytotoxic anticancer drugs were also protected against PKC-specific inhibitors. Combining PKC inhibitors with cytotoxic agents produced variable outcomes, such as increased or decreased cytotoxicity. In contrast, targeting the mitochondrial pathway of apoptosis by conditional expression of BAK reliably sensitised drug-resistant NSCLC to PKC-specific inhibitors. In conclusion, therapeutic targeting of the BCL-2 protein family in combination with a PKC-specific inhibitor such as PKC412 is a promising strategy to improve the efficacy of kinase inhibitors in the treatment of cancer.

### BRIEF DESCRIPTION OF THE DRAWINGS

FIGS. 1A-1B are graphic representations showing similar patterns of resistance of NSCLC cell lines treated with cytotoxic anticancer drugs and PKC-specific inhibitors in vitro.

FIGS. 2A-2E are graphic representations showing that combining cytotoxic anticancer drugs with PKC-specific inhibitors fails to result in predictable synergistic cytotoxicity in vitro.

FIGS. 3A-3D are graphic and pictorial representations showing that NSCLC cell lines resistant to PKC-specific inhibitors exhibit delayed release of mitochondrial cytochrome c, maintain Δψₘ, and fail to activate caspases.

FIGS. 4A-4D are pictorial and graphic representations showing that conditional expression of BAK sensitises drug-resistant NSCLC cell lines to apoptosis.

FIGS. 5A-5D are graphic representations showing that targeting mitochondrial BAK sensitises drug-resistant NSCLC cell lines to PKC412-induced apoptosis.

### DETAILED DESCRIPTION

The molecular understanding of cellular signal transduction pathways regulating survival, genetic stability, metabolic activity and proliferation has vastly increased during the past decades. Accordingly, careful analyses conducted in preclinical cancer models and in tumour samples led to the identification of specific deregulations of these pathways as contributing or even causative events during malignant transformation and cancer progression (1). Against this background, efforts are in place to develop therapies that are tailored for specific targets separating cancer cells from their non-malignant counterparts.

The BCL2 oncogene (OMIM 151430) functions as a potent suppressor of apoptosis under diverse conditions. Bcl-2 Antagonist Killer-1 (“BAK1” OMIM 600516) protein, a Bcl-2 homolog, was discovered that antagonizes Bcl-2, promotes cell death and counteracts the protection from apoptosis provided by Bcl-2. Overexpression of BAK induces rapid and extensive apoptosis of serum-deprived fibroblasts, suggesting that BAK is directly involved in activating the cell death machinery. BAK primarily enhances apoptotic cell death following an appropriate stimulus. Therefore, BAK modulators are useful in modulating apoptotic signal transduction pathways. The successful clinical application of the small drug kinase inhibitor imatinib in BCR-ABL-positive leukaemias and in gastrointestinal stromal tumours has impressively provided proof-of-principle for such a concept (2).

However, pharmacologic inhibitors of apparently less essential signal transduction pathways exhibited only minor clinical activity in unselected patient populations. Further, combining cytotoxic drugs with non-antibody inhibitors so far failed to produce improved clinical outcomes in lung cancer or colorectal cancer patients (3-6). Based on these observations we hypothesized that cancer cell death induced by pharmacologic kinase inhibitors is executed via molecular pathways distinct from those triggered by standard cytotoxic anticancer drugs. Alternatively, both pathways could converge at a common step in signal transduction, which then would constitute a strategic target for breaking drug resistance.

To this end we compared the sensitivity of a panel of well characterised NSCLC cell lines to cell death induced by the PKC-specific inhibitors staurosporine (STS), its clinically applied derivative N-benzoyl staurosporine (PKC412, Novartis Pharma), and common cytotoxic anticancer drugs. The model of PKC inhibition was selected based on PKC's role as a central mediator of a variety of signal transduction pathways that are considered to be critical for tumour growth and survival (7, 8). Despite this potentially broad therapeutic spectrum, we found that PKC-specific inhibitors failed to induce apoptosis in NSCLC cells that were also resistant to standard cytotoxic anticancer drugs. Molecular dissection revealed that functional defects at the level of the BCL-2 family proteins critically contributed to apoptosis resistance in those NSCLC. Therapeutic targeting of the mitochondrial step in apoptotic signal transduction was able to circumvent cross-resistance against PKC inhibitors and cytotoxic drugs.

During oncogenesis and tumour progression, cancer cells acquire a plethora of functional defects in tumour suppressor pathways. This is frequently achieved by mutational inactivation or loss of expression of tumour suppressive genes, or by genetic amplification and genetic deregulation of factors promoting survival or proliferation. In addition, epigenetic mechanisms were shown to contribute to the aberrant expression patterns observed in malignant phenotypes (1). Apoptosis is one of the main tumour suppressor pathways to be overcome on the road to cancerous transformation. Accordingly, inhibition of apoptosis was shown to promote tumour development in various preclinical cancer models (20-22), and defects in apoptotic signal transduction are frequently encountered in human cancers (23, 24). Besides promoting cancer development, apoptosis defects also seem to confer resistance to common cytotoxic therapies (24, 25), which still are the mainstay of cancer treatment in clinical oncology.

Recently, novel therapies have been introduced to cancer medicine that aim to specifically target tumour cells via immune-mediated mechanisms or via interference with deregulated signal transduction pathways. Successful examples of immune-mediated cancer therapies are the transfer of T-lymphocytes during or following haematopoietic stem cell transplantation for leukaemia, the administration of monoclonal antibodies such as trastuzumab or rituximab for patients with breast cancer or B-cell lymphoma, or the use of interferon-alpha in patients with malignant melanoma and high risk for relapse. Inhibitors of signal transduction that proved clinically effective include imatinib in patients with chronic myeloid leukaemia and gastrointestinal stromal tumours, bevacizumab and cetuximab in patients with colorectal cancer, erlotinib in patients with relapsed lung cancer, or sorafinib in patients with metastatic renal cell cancer. These examples have fostered the identification of a wide range of novel compounds and treatment strategies, some of which already have entered clinical development.

It remains an open question in the field whether these new modalities can in fact cure cancers resistant to conventional cytotoxic therapies. In a model of allogeneic haematopoietic stem cell transplantation, we have recently shown that genetic inhibitors of apoptotic signal transduction can confer cancer cell resistance to antigen-specific, cytotoxic T-lymphocytes in vitro and in vivo (26). This formally demonstrates that resistance factors, which protect cancer cells against standard cytotoxic therapies, may also lead to escape from immune-mediated tumour suppression.

In the present study, we extend the concept of “cross-resistance” to pharmacologic inhibitors of signal transduction. As a model, we have used NSCLC and inhibitors of PKC.

NSCLC is a highly prevalent malignancy and leader in cancer-related deaths in the Western World. Most NSCLC are diagnosed in advanced disease stages, and thus require drug and radiation therapy. Current standard therapies for advanced non-resectable NSCLC achieve clinically meaningful tumour regressions only in a minor fraction of patients. The median survival of advanced NSCLC patients treated in large clinical trials ranges from 10 to 12 months. Due to this high medical need, novel therapies including inhibitors of signal transduction pathways are heavily studied in NSCLC. So far, many efforts have focused on inhibitors of signaling via the epidermal growth factor receptors (EFGR). Compounds like gefitinib and erlotinib were shown to result in some clinical improvement, and even produced a short prolongation of median survival of patients with relapsed NSCLC (27, 28). However, when studied in large patient cohorts as first line therapy in combination with standard cytotoxic drug regimens, none of these compounds led to a clinical benefit (3-5). It was found that only patients with certain activating mutations of the EGFR have a high probability of response to treatment with gefitinib (29, 30). Unfortunately, the vast majority of NSCLC patients fail to exhibit such mutations, which poses the problem of broad clinical applicability of highly specific kinase inhibitors in NSCLC.

To the contrary, the PKC enzyme family is involved in several signal transduction pathways that may contribute to cancer development. These include mitogenic signaling via the platelet-derived growth factor (PDGF) receptor, regulation of cell cycle checkpoints at the G1 and G2 phases, and signaling via the vascular endothelial growth factor (VEGF) receptors on endothelial cells and cancer cells (7). Accordingly, PKC-specific inhibitors, such as STS or PKC412 induced cell cycle arrest or apoptosis in cancer cell lines, and exhibited antitumoral and antiangiogenic effects in a murine xenograft model of lung cancer (8, 31, 32). Oral administration of PKC412 was shown to be safe and feasible in a phase I study conducted in patients with advanced cancers (33). In addition, the safety of combining PKC412 with a standard cytotoxic regimen of CDDP/gemcitabine was established in a phase I study in patients with advanced NSCLC (34).

Against this background, we found that PKC-specific inhibitors, such as STS and PKC412, were most efficacious in those NSCLC cell lines that exhibit a good response to standard cytotoxic anticancer drugs. In contrast, drug-resistant NSCLC cell lines were also less sensitive to PKC inhibition-induced apoptosis. Unfortunately, this pattern of resistance could not be overcome by combining cytotoxic anticancer drugs with PKC inhibitors. Unlike other studies conducted in a limited number of NSCLC cell lines (32, 35), combination therapy in our hands did not generally result in synergistic cytotoxicity. Unexpectedly PKC412 even antagonized the activity of cytotoxic drugs in some models. These results should be taken into consideration when designing clinical efficacy studies of PKC inhibitors in combination with cytotoxic anticancer drugs in NSCLC and also in other malignant diseases. As of today, patient selection for such trials is usually based on the histopathological classification of tumours. All cell lines used in the present study originated from NSCLC, again demonstrating that histopathology alone is unable to discover functional heterogeneity. Moreover, the functional status of the TP53 tumour suppressor gene, as well as expression analysis of various regulators of apoptosis failed to predict the sensitivity to cytotoxic anticancer drugs as well as to PKC-specific inhibitors in vitro. In contrast, functional analyses of apoptotic signal transduction pathways revealed defects at the level of MOM permeabilisation in resistant NSCLC cell lines. Therapeutic targeting of this defect by conditional expression of pro-apoptotic BAK reliably overcame resistance to PKC inhibitors and/or standard cytotoxic drugs.

Certainly, such extensive biochemical analyses cannot be easily performed in tumour biopsies obtained from cancer patients. However, our results may have several implications on the development of strategies for the translation of novel compounds in clinical oncology. First, combining kinase inhibitors with standard cytotoxic regimens may not be informative, as the outcome of this combined treatment cannot be predicted for the heterogeneous population of patients with histopathologically classified cancers. Positive effects of the combination in some patients may be outweighed by detrimental effects in others, resulting at best in similar net outcomes following combination therapy (3-6). Secondly, the efficacy of novel targeted drugs may be hampered by the very same resistance mechanisms leading to failure of cytotoxic anticancer drugs. In the current study, this was demonstrated for defects in apoptotic signal transduction. The same may be true for defects in cell cycle regulation, or alternative death pathways. Thirdly, careful functional analyses conducted in preclinical cancer models can identify molecular targets that are strategically placed at a convergence point of several death and survival pathways.

In our present study, retroviral gene transfer and conditional expression of BAK was devised to model therapeutic modulation of such a target. Translation into clinical reality most likely requires different pharmacologic strategies, such as small compound modulators of the pro- and anti-apoptotic rheostat at the level of the BCL-2 family proteins (36,37).

The present invention relates to a method of treating solid tumors such as e.g., colorectal cancer (CRC) and non-small cell lung cancer (NSCLC) with protein kinase C inhibitor. It also relates to the use of a pharmaceutical combination of a FLT-3 kinase inhibitor and a BAK inhibitor for the treatment of the diseases or malignancies mentioned above and the use of such a pharmaceutical composition for the manufacture of a medicament for the treatment of these diseases or malignancies.

It has now surprisingly been found that FLT-3 kinase inhibitors in combination with activators of mitochondrial outer membrane permeability, such as activators of BAK, possess therapeutic properties that render them particularly useful for the treatment of e.g., non-small cell lung cancer (NSCLC).

Abbreviations

ActD—actinomycin D, CDDP—cisplatin, DOX—doxycycline, DXR—doxorubicine, EGFP—enhanced green fluorescent protein, EGFR—epidermal growth factor receptor, MOM—mitochondrial outer membrane, NSCLC—Non-small cell lung cancer, PDGF—platelet-derived growth factor, PKC—protein kinase C, PKC412-N-benzoyl staurosporine, STS—staurosporine, VEGF—vascular endothelial growth factor, VP16—etoposide.

FLT-3 Kinase Inhibitors

FLT-3 kinase inhibitors of particular interest for use in the inventive combination are staurosporine derivatives. Preferably the FLT-3 inhibitor is N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-Im]pyrrolo[3,4j][1,7]benzodiazonin-11-yl]-N-methylbenzamide of formula I:

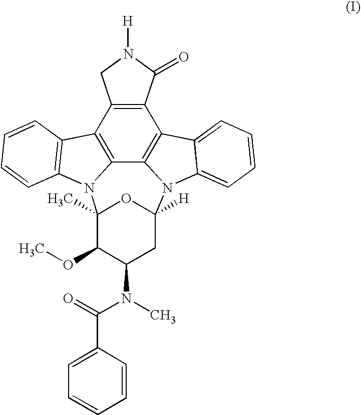

or a salt thereof, including especially a pharmaceutically acceptable salt. The compound of formula I is also known as MIDOSTAURIN [International Nonproprietary Name] or PKC412. PKC412 is a derivative of the naturally occurring alkaloid staurosporine

In alternative embodiments, suitable Flt-3 inhibitors include e.g.: compounds as disclosed in WO 03/037347, e.g. staurosporine derivatives of formula (II) or (III):

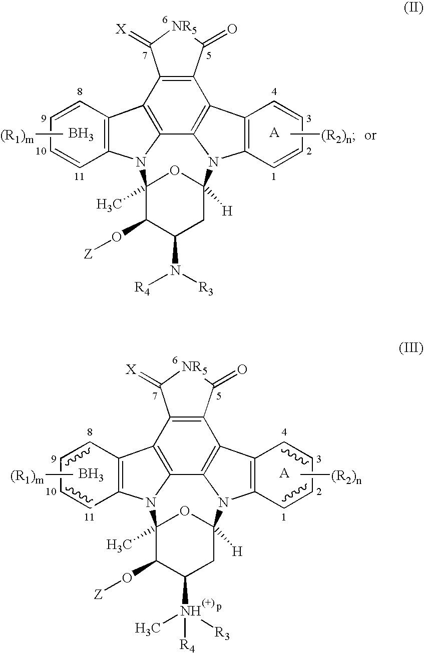

wherein the compound (III) is the partially hydrogenated derivative of compound (II); or staurosporine derivatives of formula (IV) or (V) or (VI) or (VII):

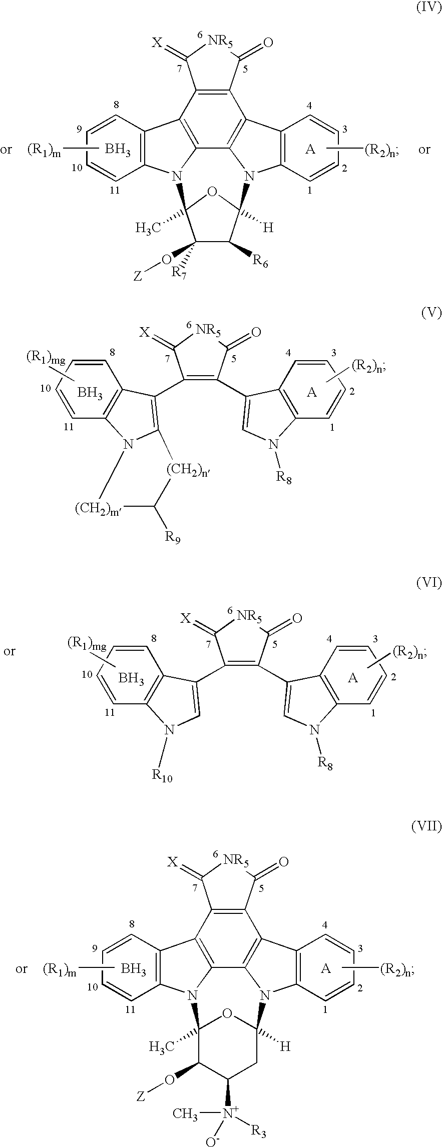

wherein R₁ and R₂, are, independently of one another, unsubstituted or substituted alkyl, hydrogen, halogen, hydroxy, etherified or esterified hydroxy, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
n and m are, independently of one another, a number from and including 0 to and including 4;
n′ and m′ are, independently of one another, a number from and including 0 to and including 4;
R₃, R₄, R₈ and R₁₀ are, independently of one another, hydrogen, —O⁻, acyl with up to 30 carbon atoms, an aliphatic, carbocyclic, or carbocyclic-aliphatic radical with up to 29 carbon atoms in each case, a heterocyclic or heterocyclic-aliphatic radical with up to 20 carbon atoms in each case, and in each case up to 9 heteroatoms, an acyl with up to 30 carbon atoms, wherein R₄ may also be absent;
or if R₃ is acyl with up to 30 carbon atoms, R₄ is not an acyl;
p is 0 if R₄ is absent, or is 1 if R₃ and R₄ are both present and in each case are one of the aforementioned radicals;
R₅ is hydrogen, an aliphatic, carbocyclic, or carbocyclic-aliphatic radical with up to 29 carbon atoms in each case, or a heterocyclic or heterocyclic-aliphatic radical with up to 20 carbon atoms in each case, and in each case up to 9 heteroatoms, or acyl with up to 30 carbon atoms;
R₇, R₆ and R₉ are acyl or -(lower alkyl)-acyl, unsubstituted or substituted alkyl, hydrogen, halogen, hydroxy, etherified or esterified hydroxy, amino, mono- or disubstituted amino, cyano, nitro, mercapto, substituted mercapto, carboxy, carbonyl, carbonyldioxy, esterified carboxy, carbamoyl, N-mono- or N,N-di-substituted carbamoyl, sulfo, substituted sulfonyl, aminosulfonyl or N-mono- or N,N-di-substituted aminosulfonyl;
X stands for 2 hydrogen atoms; for 1 hydrogen atom and hydroxy; for O; or for hydrogen and lower alkoxy;
Z stands for hydrogen or lower alkyl;
and either the two bonds characterised by wavy lines are absent in ring A and replaced by 4 hydrogen atoms, and the two wavy lines in ring B each, together with the respective parallel bond, signify a double bond;
or the two bonds characterised by wavy lines are absent in ring B and replaced by a total of 4 hydrogen atoms, and the two wavy lines in ring A each, together with the respective parallel bond, signify a double bond;
or both in ring A and in ring B all of the 4 wavy bonds are absent and are replaced by a total of 8 hydrogen atoms;
or a salt thereof, if at least one salt-forming group is present.

The general terms and definitions used hereinbefore and hereinafter preferably have the meanings for the staurosporine derivatives as provided in WO 03/037347, which is incorporated herein by reference in its entirety. However, where discrepancies appear between WO 03/037347 and the instant disclosure, the instant disclosure shall govern.

By their nature, the compounds of the invention may be present in the form of pharmaceutically, i.e. physiologically, acceptable salts, provided they contain salt-forming groups. For isolation and purification, pharmaceutically unacceptable salts may also be used. For therapeutic use, only pharmaceutically acceptable salts are used, and these salts are preferred.

Thus, compounds of formula I having free acid groups, for example a free sulfo, phosphoryl or carboxyl group, may exist as a salt, preferably as a physiologically acceptable salt with a salt-forming basic component. These may be primarily metal or ammonium salts, such as alkali metal or alkaline earth metal salts, for example sodium, potassium, magnesium or calcium salts, or ammonium salts with ammonia or suitable organic amines, especially tertiary monoamines and heterocyclic bases, for example triethylamine, tri-(2-hydroxyethyl)-amine, N-ethylpiperidine or N,N′-dimethylpiperazine.

Compounds of the invention having a basic character may also exist as addition salts, especially as acid addition salts with inorganic and organic acids, but also as quaternary salts. Thus, for example, compounds which have a basic group, such as an amino group, as a substituent may form acid addition salts with common acids. Suitable acids are, for example, hydrohalic acids, e.g., hydrochloric and hydrobromic acid, sulfuric acid, phosphoric acid, nitric acid or perchloric acid, or aliphatic, alicyclic, aromatic or heterocyclic carboxylic or sulfonic acids, such as formic, acetic, propionic, succinic, glycolic, lactic, malic, tartaric, citric, fumaric, maleic, hydroxymaleic, oxalic, pyruvic, phenylacetic, benzoic, p-aminobenzoic, anthranilic, p-hydroxybenzoic, salicylic, p-aminosalicylic acid, pamoic acid, methanesulfonic, ethanesulfonic, hydroxyethanesulfonic, ethylenedisulfonic, halobenzenesulfonic, toluenesulfonic, naphthalenesulfonic acids or sulfanilic acid, and also methionine, tryptophan, lysine or arginine, as well as ascorbic acid.

In view of the close relationship between the compounds (especially of formula I) in free form and in the form of their salts, including those salts that can be used as intermediates, for example in the purification or identification of the novel compounds, and of their solvates, any reference hereinbefore and hereinafter to the free compounds is to be understood as referring also to the corresponding salts, and the solvates thereof, for example hydrates, as appropriate and expedient.

STAUROSPORINE DERIVATIVES and their manufacturing process have been specifically described in many prior documents, well known by one skilled in the art.

Compounds of formula I and their manufacturing processes have specifically been described in the European patents No. 0 296 110 published on Dec. 21, 1988, as well as in U.S. Pat. No. 5,093,330 published on Mar. 3, 1992, and Japanese Patent No. 2 708 047, each of which are incorporated herein by reference.

In each case where citations of patent applications or scientific publications are given in particular for the STAUROSPORINE DERIVATIVE compounds, the subject-matter of the final products, the pharmaceutical preparations and the claims are hereby incorporated into the present application by reference to these publications.

The structure of the active agents identified by code nos., generic or trade names may be taken from the actual edition of the standard compendium “The Merck Index” or from databases, e.g., Patents International (e.g., IMS World Publications). The corresponding content thereof is hereby incorporated by reference.

BAK Activators

BAK modulators are useful in modulating apoptotic signal transduction pathways. BAK activators enhance apoptotic cell death and counteract the anti-apoptotic effects of BCL2. BAK activators include but are not limited to BCL-2/BCL-XL inhibitors. Examples of Bcl-2/Bcl-XL inhibitory compounds include but are not limited to anti-Bcl-2/Bcl-XL antibodies, RNAi constructs targeting either Bcl-2 or Bcl-XL, hydrocarbon-stapled BH3 helix peptides and chemical inhibitors such as N-{4-[4-(4′-Chloro-biphenyl-2-ylmethyl)-piperazin-1-yl]-benzoyl}-4-(3-dimethylamino-1-phenylsulfanylmethyl-propylamino)-3-nitro-benzenesulfonamide (Abbott compound ABT-737) (36, 37). Apoptosis therapies including additional BAK activators were recently reviewed (40).

Therapeutics, Medicaments and Methods of Use

The present invention in particular provides a method of treating non-small cell lung cancer (NSCLC), comprising administering to a mammal in need of such a treatment a therapeutically effective amount of a FLT-3 kinase inhibitor, either in free form or in the form of a pharmaceutically acceptable salt or prodrug. A preferred FLT-3 kinase inhibitor is PKC412.

Preferably the instant invention provides a method for treating mammals, especially humans, suffering from non-small cell lung cancer (NSCLC) comprising administering to a mammal in need of such treatment a therapeutically effective amount of a FLT-3 inhibitor, or a pharmaceutically acceptable salt or prodrug thereof. A preferred FLT-3 kinase inhibitor is PKC412.

In another embodiment, the instant invention relates to the use of a FLT-3 kinase inhibitor, in free form or in the form of a pharmaceutically acceptable salt or prodrug, for treating NSCLC. A preferred FLT-3 kinase inhibitor is PKC412.

In a further embodiment, the instant invention relates to the use of a FLT-3 kinase inhibitor, in free form or in form of a pharmaceutically acceptable salt or prodrug, for the preparation of a pharmaceutical composition for treating NSCLC. A preferred FLT-3 kinase inhibitor is PKC412.

The precise dosage of the FLT-3 inhibitor and the compound to be employed for treating the diseases and conditions mentioned herein depends upon several factors including the host, the nature and the severity of the condition being treated, the mode of administration. However, in general, satisfactory results are achieved when the FLT-3 inhibitor is administered parenterally, e.g., intraperitoneally, intravenously, intramuscularly, subcutaneously, intratumorally, or rectally, or enterally, e.g., orally, preferably intravenously or, preferably orally, intravenously at a daily dosage of 0.1 to 10 mg/kg body weight, preferably 1 to 5 mg/kg body weight. In human trials a total dose of 225 mg/day was most presumably the Maximum Tolerated Dose (MTD). A preferred intravenous daily dosage is 0.1 to 10 mg/kg body weight or, for most larger primates, a daily dosage of 200-300 mg. A typical intravenous dosage is 3 to 5 mg/kg, three to five times a week.

Most preferably, the FLT-3 inhibitors, especially MIDOSTAURIN, are administered orally, by dosage forms such as microemulsions, soft gels or solid dispersions in dosages up to about 250 mg/day, in particular 225 mg/day, administered once, twice or three times daily.

Usually, a small dose is administered initially and the dosage is gradually increased until the optimal dosage for the host under treatment is determined. The upper limit of dosage is that imposed by side effects and can be determined by trial for the host being treated.

Combined Treatment

In one aspect, the present invention also relates to a combination, such as a combined preparation or a pharmaceutical composition, which comprises (a) a FLT-3 inhibitor, especially the FLT-3 inhibitors specifically mentioned hereinbefore, in particular those mentioned as being preferred, and in the treatment of a cytotoxic drug-resistance NSCLC (b) an activator of mitochondrial outer membrane permeabilization, such as an activator of BAK; or alternatively in the treatment of a cytotoxic drug-sensitive NSCLC (b′) a topoisomerase inhibitor; in which the active ingredients (a) and either (b) or (b′) (hereinafter “(b or b′)”) are present in each case in free form or in the form of a pharmaceutically acceptable salt, for simultaneous, concurrent, separate or sequential use.

The term “a combined preparation” defines especially a “kit of parts” in the sense that the combination partners (a) and (b or b′) as defined above can be dosed independently or by use of different fixed combinations with distinguished amounts of the combination partners (a) and (b or b′), i.e., simultaneously, concurrently, separately or sequentially. The parts of the kit of parts can then, e.g., be administered simultaneously or chronologically staggered, that is at different time points and with equal or different time intervals for any part of the kit of parts. The ratio of the total amounts of the combination partner (a) to the combination partner (b or b′) to be administered in the combined preparation can be varied, e.g., in order to cope with the needs of a patient sub-population to be treated or the needs of the single patient which different needs can be due to the particular disease, severity of the disease, age, sex, body weight, etc. of the patients.

Suitable clinical studies are, for example, open label, dose escalation studies in patients with proliferative diseases. Such studies prove in particular the synergism of the active ingredients of the combination of the invention. The beneficial effects on NSCLC can be determined directly through the results of these studies which are known as such to a person skilled in the art. Such studies are, in particular, suitable to compare the effects of a monotherapy using the active ingredients and a combination of the invention. Preferably, the dose of agent (a) is escalated until the Maximum Tolerated Dosage is reached, and agent (b or b′) is administered with a fixed dose. Alternatively, the agent (a) is administered in a fixed dose and the dose of agent (b or b′) is escalated. Each patient receives doses of the agent (a) either daily or intermittent. The efficacy of the treatment can be determined in such studies, e.g., after 12, 18 or 24 weeks by evaluation of symptom scores every 6 weeks.

The administration of a pharmaceutical combination of the invention results not only in a beneficial effect, e.g., a synergistic therapeutic effect, e.g., with regard to alleviating, delaying progression of or inhibiting the symptoms, but also in further surprising beneficial effects, e.g., fewer side-effects, an improved quality of life or a decreased morbidity, compared with a monotherapy applying only one of the pharmaceutically active ingredients used in the combination of the invention.

A further benefit is that lower doses of the active ingredients of the combination of the invention can be used, for example, that the dosages need not only often be smaller but are also applied less frequently, which may diminish the incidence or severity of side-effects. This is in accordance with the desires and requirements of the patients to be treated.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

It is one objective of this invention to provide a pharmaceutical composition comprising a quantity, which is jointly therapeutically effective at targeting or preventing proliferative diseases a combination of the invention. In this composition, agent (a) and agent (b or b′) may be administered together, one after the other or separately in one combined unit dosage form or in two separate unit dosage forms. The unit dosage form may also be a fixed combination.

The pharmaceutical compositions for separate administration of agent (a) and agent (b or b′) or for the administration in a fixed combination, i.e. a single galenical composition comprising at least two combination partners (a) and (b or b′), according to the invention may be prepared in a manner known per se and are those suitable for enteral, such as oral or rectal, and parenteral administration to mammals (warm-blooded animals), including humans, comprising a therapeutically effective amount of at least one pharmacologically active combination partner alone, e.g., as indicated above, or in combination with one or more pharmaceutically acceptable carriers or diluents, especially suitable for enteral or parenteral application.

Suitable pharmaceutical compositions contain, for example, from about 0.1% to about 99.9%, preferably from about 1% to about 60%, of the active ingredient(s). Pharmaceutical preparations for the combination therapy for enteral or parenteral administration are, for example, those in unit dosage forms, such as sugar-coated tablets, tablets, capsules or suppositories, or ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each of the combination partner of the combination of the invention may be administered simultaneously or sequentially and in any order, and the components may be administered separately or as a fixed combination. For example, the method of preventing or treating proliferative diseases according to the invention may comprise (i) administration of the first agent (a) in free or pharmaceutically acceptable salt form and (ii) administration of an agent (b or b′) in free or pharmaceutically acceptable salt form, simultaneously or sequentially in any order, in jointly therapeutically effective amounts, preferably in synergistically effective amounts, e.g., in daily or intermittently dosages corresponding to the amounts described herein. The individual combination partners of the combination of the invention may be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of the combination partners employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration and the renal and hepatic function of the patient. A clinician or physician of ordinary skill can readily determine and prescribe the effective amount of the single active ingredients required to alleviate, counter or arrest the progress of the condition. Optimal precision in achieving concentration of the active ingredients within the range that yields efficacy without toxicity requires a regimen based on the kinetics of the active ingredients' availability to target sites.

Daily dosages for agent (a) or (b or b′) or will, of course, vary depending on a variety of factors, for example the compound chosen, the particular condition to be treated and the desired effect. In general, however, satisfactory results are achieved on administration of agent (a) at daily dosage rates of the order of ca. 0.03 to 5 mg/kg per day, particularly 0.1 to 5 mg/kg per day, e.g., 0.1 to 2.5 mg/kg per day, as a single dose or in divided doses. Agent (a) and agent (b or b′) may be administered by any conventional route, in particular enterally, e.g., orally, e.g., in the form of tablets, capsules, drink solutions or parenterally, e.g., in the form of injectable solutions or suspensions. Suitable unit dosage forms for oral administration comprise from ca. 0.02 to 50 mg active ingredient, usually 0.1 to 30 mg, e.g., agent (a) or (b or b′), together with one or more pharmaceutically acceptable diluents or carriers therefore.

Agent (b or b′) may be administered to a human in a daily dosage range of 0.5 to 1000 mg. Suitable unit dosage forms for oral administration comprise from ca. 0.1 to 500 mg active ingredient, together with one or more pharmaceutically acceptable diluents or carriers therefore.

The administration of a pharmaceutical combination of the invention results not only in a beneficial effect, e.g., a synergistic therapeutic effect, e.g., with regard to inhibiting the unregulated proliferation of or slowing down the progression of NSCLC, but also in further surprising beneficial effects, e.g., less side-effects, an improved quality of life or a decreased morbidity, compared to a monotherapy applying only one of the pharmaceutically active ingredients used in the combination of the invention.

A further benefit is that lower doses of the active ingredients of the combination of the invention can be used, for example, that the dosages need not only often be smaller but are also applied less frequently, or can be used in order to diminish the incidence of side-effects. This is in accordance with the desires and requirements of the patients to be treated.

The (a) and the (b or b′) compound may be combined with one or more pharmaceutically acceptable carriers and, optionally, one or more other conventional pharmaceutical adjuvants and administered enterally, e.g., orally, in the form of tablets, capsules, caplets, etc. or parenterally, e.g., intraperitoneally or intravenously, in the form of sterile injectable solutions or suspensions. The enteral and parenteral compositions may be prepared by conventional means.

The infusion solutions according to the present invention are preferably sterile. This may be readily accomplished, e.g., by filtration through sterile filtration membranes. Aseptic formation of any composition in liquid form, the aseptic filling of vials and/or combining a pharmaceutical composition of the present invention with a suitable diluent under aseptic conditions are well known to the skilled addressee.

The FLT-3 inhibitors may be formulated into enteral and parenteral pharmaceutical compositions containing an amount of the active substance that is effective for treating the diseases and conditions named hereinbefore, such compositions in unit dosage form and such compositions comprising a pharmaceutically acceptable carrier.

The described pharmaceutical compositions comprise a solution or dispersion of compounds of formula I such as MIDOSTAURIN in a saturated polyalkylene glycol glyceride, in which the glycol glyceride is a mixture of glyceryl and polyethylene glycol esters of one or more C8-C18 saturated fatty acids.

Preferably, there is at least one beneficial effect, e.g., a mutual enhancing of the effect of the first and second active ingredient, in particular a synergism, e.g., a more than additive effect, additional advantageous effects, less side effects, a combined therapeutic effect in a otherwise non-effective dosage of one or both of the first and second active ingredient, and especially a strong synergism the active ingredients.

The efficacy of PKC412 for the treatment of NSCLC is illustrated by the results of the following examples. These examples illustrate the invention without in any way limiting its scope.

### EXAMPLES

### Example 1

### Cell Lines and Vectors

NSCLC cell lines known in the art were obtained. Unless otherwise specified, NSCLC cells were grown on tissue culture dishes (BD Falcon) in Dulbecco's Modified Eagle Medium supplemented with 10% fetal bovine serum, glucose, L-glutamine and penicillin/streptomycin in a humidified atmosphere at 5% CO₂. NSCLC cells conditionally expressing transgenic BAK were obtained using the BD RevTet-On vector system (BD Clontech). A BamHI fragment encoding the full length human BAK cDNA was generated by PCR, confirmed by sequencing, and cloned into the pRevTRE vector. A retroviral BCL-XL expression vector has been described previously (26). Replication-defective retroviral virions were produced by standard calcium phosphate transfection in the FNX ampho packaging cell line (a gift from Dr G. P. Nolan, Stanford). Transductions were performed using filtered supernatants, and populations were selected with hygromycin B and puromycin in the absence of tetracycline, or were obtained by fluorescence activated cell sorting (Coulter) of EGFP-positive cells.

### Example 2

### Apoptosis Assays

Quantitation of cells with fragmented DNA, activated caspases, lost mitochondrial transmembrane potential, and measurements of cell cycle distribution were performed by flow cytometry (Coulter) as previously described (26,38,39). N-benzoyl staurosporine (PKC412) was obtained from Novartis Pharma, Basel, Switzerland, and zVAD-fmk was obtained from ICN. All other drugs were purchased from Sigma.

### Example 3

### Immunoblotting

Immunoblotting and cell fractionation was performed as described previously (38, 39) using primary antibodies against caspase-9 (Chemicon), caspase-3, BCL-XL, cytochrome c (BD Pharmingen), BAX, BAK, PARP (Upstate), AKT, phospho-AKT, GSK-3beta, phospho-GSK3beta (Cell Signaling), and actin (ICN).

### Example 4

### Resistance of NSCLC Cell Lines

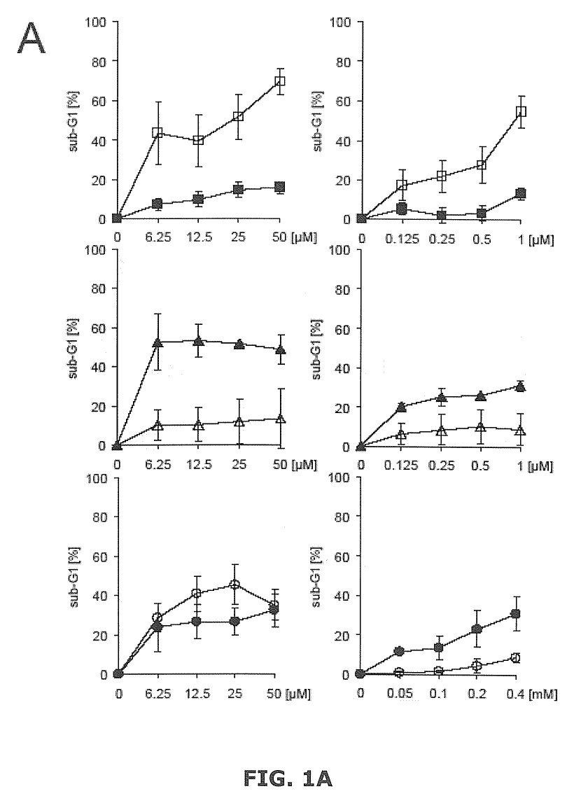

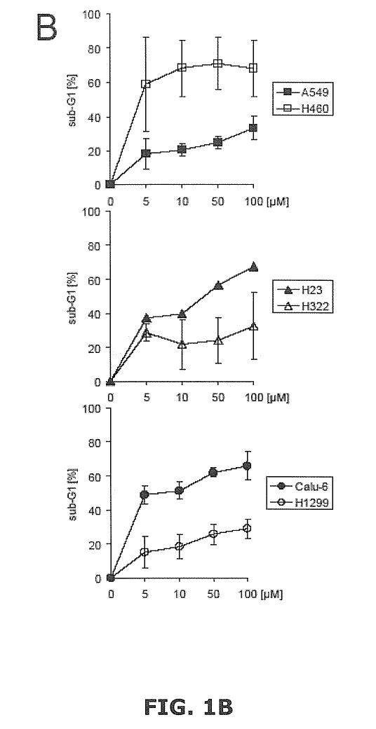

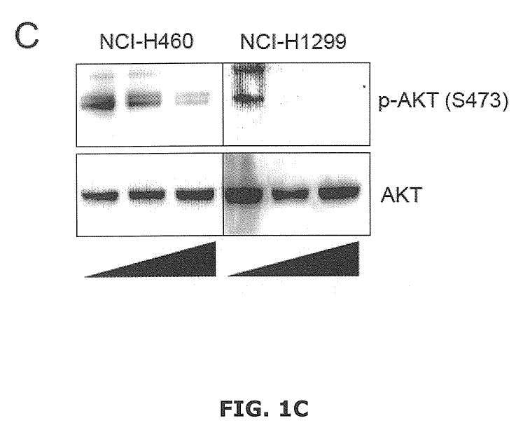

In FIG. 1A TP53-proficient NCI-H460 (open boxes) and A549 (closed boxes), TP53 mutant NCI-H322 (open triangles) and NCI-H23 (closed triangles), and TP53-deficient NCI-H1299 (open circles) and Calu-6 (closed circles) NSCLC cells were treated with etoposide (left column), cisplatin (right column, bottom panel, or doxorubicine (right column, top and center panel) at the indicated doses. After 48 hours, the percentage of cells with subdiploid DNA content (sub-G1) was measured by flow cytometry as an indicator of apoptosis. In FIG. 1B the same NSCLC cell lines as in FIG. 1A were treated with escalating doses of the PKC-specific inhibitor PKC412. The percentages of cells with subdiploid DNA content was quantified by flow cytometry after 48 hours of treatment. Mean values ±standard deviations (SD) of at least three independent experiments are given. In FIG. 1C drug-sensitive NCI-H460 cells, and drug-resistant NCI-H1299 cells were pretreated with PKC412 (1 to 10 μM) or DMSO for 2 hours, followed by stimulation with PMA (1 μM) for 10 minutes. Whole cell extracts were analysed by immunoblotting using the indicated primary antibodies.

### Example 5

### Drug synergy Analysis

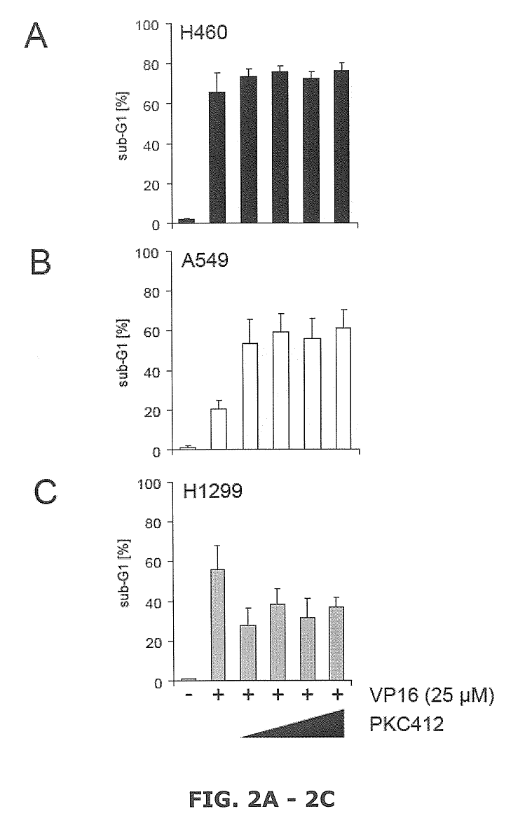

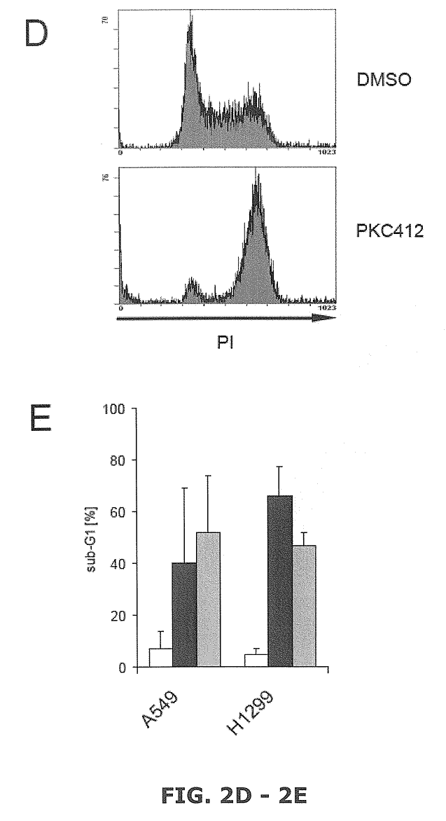

TP53-proficient NCI-H460 cells (FIG. 2A, black bars), A549 cells (FIG. 2B, white bars), and TP53-deficient NCI-H1299 cells (FIG. 2C, grey bars) were simultaneously treated with 25 μM etoposide and escalating doses of PKC412 (0, 5, 10, 50, 100 μM), and cells with subdiploid DNA content were quantified after 48 hours. Mean values +SD of at least three independent experiments are given. In FIG. 2D cell cycle distribution of NCI-H1299 treated with DMSO or 50 μM PKC 412 for 24 hours. In FIG. 2E A549 and NCI-H1299 cells were first treated with 25 μM etoposide for 24 hours followed by addition of 50 μM PKC412 for another 24 hours (black bars). Alternatively, cells were treated with 50 μM PKC412 for 24 hours followed by the addition of 25 μM etoposide for another 24 hours (grey bars). After 48 hours, the fraction of cells with subdiploid DNA content (sub-G1) was quantified by flow cytometry. DMSO-treated cells (white bars) served as negative controls. Mean values +SD of at least three independent experiments are given.

### Example 6

### Mitochondrial Function

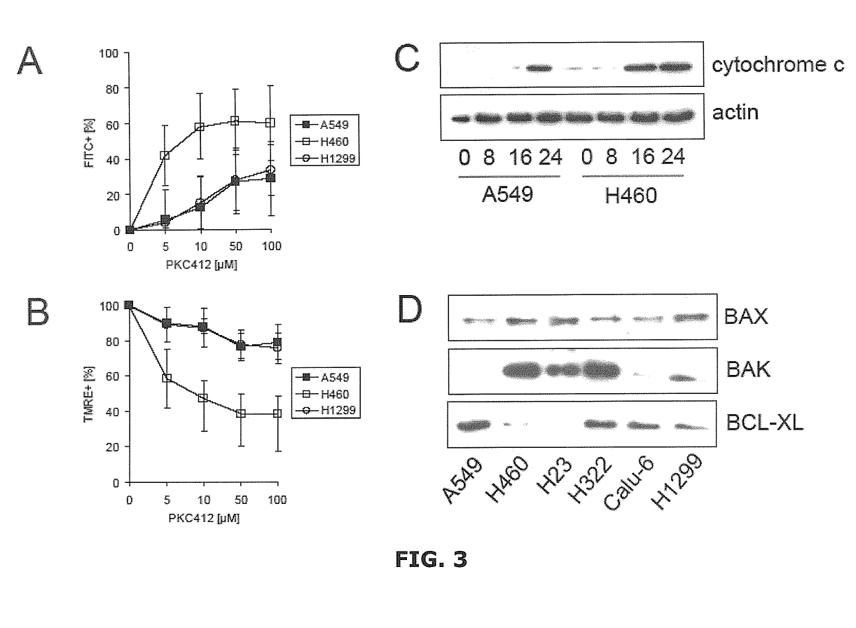

In FIG. 3A NCI-H460 (open boxes), A549 (closed boxes) and NCI-H1299 (open circles) NSCLC cells were treated with the indicated doses of PKC412. After 48 hours, cells were stained with the fluorescent caspase substrate FITC-VAD (Oncogene), and the fraction of FITC-positive cells with activated caspases (FITC+) was measured by flow cytometry. In FIG. 3B NCI-H460 (open boxes), A549 (closed boxes) and NCI-H1299 (open circles) NSCLC cells were treated with the indicated doses of PKC412. After 48 hours, cells were stained with the mitochondrial dye tetramethylrhodamine ethylester (TMRE, Molecular Probes), and the fraction of TMRE-positive cells with preserved mitochondrial transmembrane potential Δψₘ (TMRE+) was quantified by flow cytometry. Mean values ±SD of at least three independent experiments are given. In FIG. 3C NCI-H460 and A549 NSCLC cells were treated with 25 μM etoposide, and cytosolic fractions were obtained at the indicated time points. The release of mitochondrial cytochrome c into the cytosol was detected by immunoblotting using a cytochrome c-specific primary antibody. In FIG. 3D whole cell extracts were prepared from TP53-proficient A549 and NCI-H460, TP53 mutant NCI-H23 and NCI-H322, and TP53-deficient Calu-6 and NCI-H1299 NSCLC cells. The constitutive expression of BAX, BAK and BCL-XL was detected by immunoblotting.

### Example 7

### Conditional BAK Expression

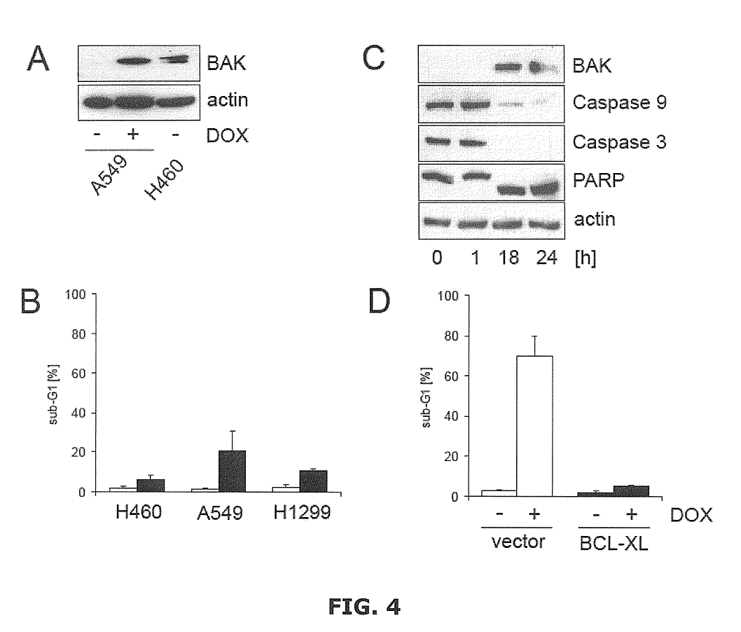

In FIG. 4A A549 cells expressing BAK under the control of a tetracycline-regulated promoter were grown in the absence (−) or presence (+) of doxycycline (DOX). Whole cell extracts were prepared 24 hours after induction of DOX, and were analysed for BAK expression by immunoblotting. Cell extracts from NCI-H460 cells served as control for endogenous expression levels of BAK. In FIG. 4B NCI-H460, A549 and NCI-H1299 NSCLC cells expressing BAK under the control of a tetracycline-regulated promoter were grown in the absence (white bars) or presence (black bars) of DOX, and cells with subdiploid DNA content (sub-G1) were quantified by flow cytometry after 24 hours. Mean values +SD of three independent experiments are shown. In FIG. 4C A549 cells expressing BAK under the control of a tetracycline-regulated promoter were treated with 25 μM etoposide in the presence of DOX, and whole cell extracts were obtained at the indicated time points. The expression of BAK, and the cleavage of caspase-9, caspase-3, and the caspase substrate PARP were detected by immunoblotting. In FIG. 4D A549 cells expressing BAK under the control of a tetracycline-regulated promoter were transduced to express BCL-XL (black bars) or a control vector (white bars) in conjunction with EGFP, and EGFP-positive populations were selected by fluorescence activated cell sorting. BAK expression was induced by the addition of DOX, and the fractions of cells with subdiploid DNA content were quantified by flow cytometry after 48 hours. Mean values +SD of three independent experiments are shown.

### Example 8

### Targeting Mitochondrial BAK

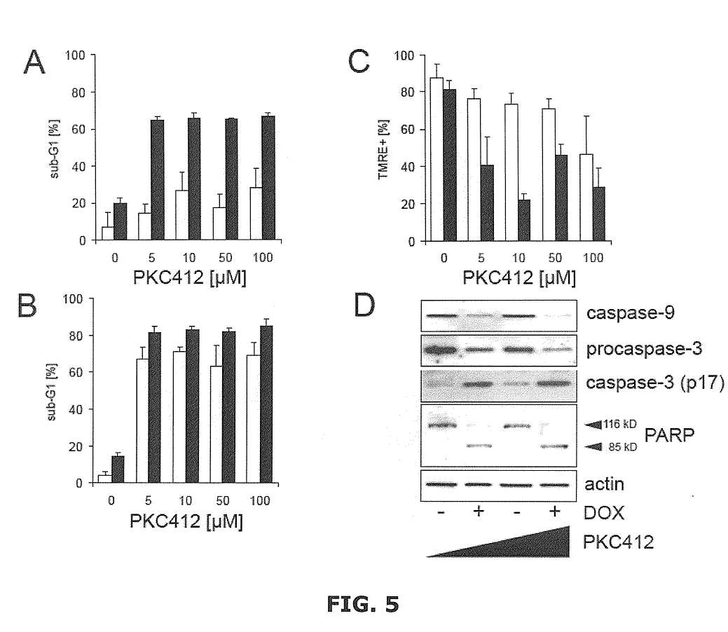

Drug-resistant A549 (FIG. 5A) and drug-sensitive NCI-H460 (FIG. 5B) cells expressing BAK under the control of a tetracycline-regulated promoter were treated with escalating doses of PKC412 in the absence (white bars) or presence (black bars) of DOX to induce BAK expression. Cells with subdiploid DNA content (sub-G1) were quantified by flow cytometry after 48 hours. Mean values +SD of at least three independent experiments are given. In FIG. 5C drug-resistant NCI-H1299 cells expressing BAK under the control of a tetracycline-regulated promoter were treated with escalating doses of PKC412 in the absence (white bars) or presence (black bars) of DOX to induce BAK expression. Cells that maintained Δψₘ (TMRE+) were quantified by TMRE-staining and flow cytometry after 48 hours. Mean values ±SD of at least three independent experiments are given. In FIG. 5D A549 cells expressing BAK under the control of a tetracycline-regulated promoter were treated with increasing doses of PKC412 (1 to 10 μM) in the absence or presence of DOX to induce BAK expression. Whole cell extracts were obtained at 24 hours, and the cleavage of caspase-9, caspase-3 and the caspase substrate PARP was detected by immunoblotting.

### Example 9

### Similar Patterns of Resistance to Protein Kinase C-Specific Inhibitors and Cytotoxic Anticancer Drugs in NSCLC

To study a possible contribution of defects in the core apoptotic machinery to drug resistance in NSCLC, three pairs of cell lines that are either proficient (A549, NCI-H460), deficient (NCI-H1299, Calu-6), or mutant (NCI-H23, NCI-H322) for the TP53 tumour suppressor gene, were analysed. Using a panel of clinically applied cytotoxic anticancer drugs including doxorubicine (DXR), cisplatin (CDDP), paclitaxel, actinomycin D (actD) and etoposide (VP16), we found a similar pattern of resistance of these cell lines that was independent of the respective cytotoxic agent (FIG. 1 A, and not shown). These results confirmed that the p53 status is a poor predictor of sensitivity to cytotoxic therapies in NSCLC.

As growth factor deprivation can induce apoptosis by mechanisms distinct from DNA damage-triggered cell death, we reasoned that inhibitors of growth factor signaling would be capable of eliminating cancer cells resistant to such cytotoxic therapies. To this end, NSCLC cell lines were treated with the PKC-specific inhibitors staurosporine and its clinically applied derivative PKC412. Interestingly, cell lines that were protected against apoptosis induced by cytotoxic drugs also showed reduced sensitivity to the PKC-specific inhibitors (FIG. 1 B and not shown). This was not explained by differences in target molecule inhibition, as PKC412 effectively reduced the phosphorylation of downstream targets of PKC signal transduction (9), such as protein kinase B/AKT and glycogen synthase kinase 3-beta, in drug-resistant and drug-sensitive cell lines (FIG. 1 C and not shown). Hence, resistance to apoptosis induced by cytotoxic anticancer drugs and inhibitors of PKC seemed to be determined by a common defect in the apoptotic signal transduction pathway.

### Example 10

### Combining PKC412 with Cytotoxic Anticancer Drugs in NSCLC Produced Variable Outcomes

To explore whether synergistic or additive effects of a combined treatment with PKC412 and cytotoxic anticancer drugs can overcome drug resistance in NSCLC, we first measured the induction of apoptosis following simultaneous incubation with a fixed dose of the topoisomerase inhibitor VP16 and increasing doses of PKC412. In sensitive cancer cell lines, such as NCI-H460, PKC412 resulted in no further increase in apoptosis as compared to VP16 alone (FIG. 2 A). Interestingly, divergent results were obtained in drug-resistant NSCLC cell lines. While combined treatment with PKC412 and VP16 produced additive cytotoxicity in A549 cells (FIG. 2 B), treatment with PKC412 actually protected NCI-H1299 cells against VP16-induced apoptosis (FIG. 2 C). To further delineate the influence of timing and sequence of the application of PKC412 and VP16, cells were either pre-treated with VP16 or PKC412 for 24 hours, followed by addition of the alternative drug for another 24 hours. Pre-treatment with PKC412 resulted in a cell cycle arrest in the G2/M-phase, which most likely is explained by inhibition of CDK1 activity (10) (FIG. 2 D). Interestingly, in NCI-H1299 cells this G2/M arrest reduced the amount of apoptosis induced by VP16 given subsequently to PKC412 (FIG. 2 E). In contrast, the amount of apoptosis found in A549 pretreated with PKC412 did not significantly differ to the one observed following pre-treatment with VP16 (FIG. 2 E).

### Example 11

### Defects in the Mitochondrial Pathway of Caspase Activation in NSCLC Cells Resistant to PKC412

Apoptosis induced by DNA damaging agents and growth factor withdrawal proceeds predominantly via the mitochondrial pathway of caspase activation (11,12). To further dissect the mechanism of resistance to PKC-specific inhibitors in the NSCLC cell lines, we analysed several steps of this apoptotic signal transduction pathway. Resistant NSCLC cell lines consistently showed reduced caspase activation and preserved mitochondrial transmembrane potential (“Δψₘ”) following treatment with PKC-specific inhibitors or cytotoxic anticancer drugs (FIG. 3 A, B and not shown). Also, the release of mitochondrial cytochrome c into the cytoplasm was delayed and reduced in drug-resistant NSCLC cell lines (FIG. 3 C). These results pointed at a block in apoptotic signal transduction at the level of the BCL-2 family proteins. To this end, we studied the constitutive expression of the essential pro-apoptotic BH1-2-3 proteins BAX and BAK, and the anti-apoptotic protein BCL-XL in NSCLC cell lines. While BAX was consistently expressed in all 6 cells lines, the protein levels of BAK and BCL-XL showed some degree of variation (FIG. 3 D). However, none of these factors convincingly explained the pattern of resistance observed in the NSCLC cell lines.

### Example 12

### Inducible Expression of BAK Sensitized Resistant NSCLC Cells to PKC412-Mediated Apoptosis

Based on our previous results, we reasoned that therapeutic targeting of proapoptotic BCL-2 family proteins would be able to overcome the functional block in caspase activation observed in drug-resistant NSCLC cell lines. A pivotal step in this pathway is the permeabilisation of the mitochondrial outer membrane (MOM), which is executed by the proapoptotic BCL-2 proteins BAX and BAK (13). Overexpression studies have shown that both molecules can directly induce MOM permeabilisation and apoptosis (14-17). In a physiological context, BAX and BAK are negatively regulated by anti-apoptotic BCL-2 proteins, such as BCL-XL, MCL-1 or BCL-2. Direct or indirect positive regulation of BAX and BAK is achieved by the group of BH3-only proteins, including but not limited to BID and BIM, or PUMA, NOXA, BAD and others (18,19).

To study the pharmacological modulation of BAK, which is constitutively targeted to the mitochondria, we generated a retroviral vector enabling conditional expression of the human BAK cDNA. In this system, the expression of transgenic BAK is induced at the transcriptional level by the addition of doxycycline (DOX). The high transduction efficacies achieved with this retroviral vector system allowed us to assess populations of NSCLC cell lines. This is a better reflection of a pharmacologic treatment of a tumour than studying single cell clones. Moreover, the expression levels of transgenic BAK in these populations did not exceed levels of endogenous BAK observed in some NSCLC cell lines (FIG. 4 A).

Inducing the expression of transgenic BAK resulted in some degree of apoptosis in drug-resistant NSCLC cell lines (FIG. 4 B). Apoptosis facilitated by transgenic BAK was accompanied by the cleavage and activation of caspases and caspase substrates (FIG. 4 C), loss of Δψₘ, and was inhibited by the expression of BCL-XL or the broad-spectrum caspase inhibitor zVAD-fmk (FIG. 4 D and not shown). These results confirm that transgenic BAK acts like its physiologic counterpart in this experimental system.

Interestingly, conditionally expressed BAK effectively sensitised drug-resistant NSCLC cell lines to apoptosis induced by PKC-specific inhibitors or cytotoxic anticancer drugs (FIG. 5 A, C and not shown). This was explained by caspase activation following treatment with PKC-specific inhibitors only in the presence, but not in the absence of DOX in these cell lines (FIG. 5 D). In contrast, inducing BAK expression in drug-sensitive NSCLC cells only marginally increased the amount of apoptosis observed after treatment with PKC-specific inhibitors (FIG. 5 B).

### REFERENCES

1. Hanahan D, Weinberg R A (2000) The Hallmarks of Cancer. Cell 100: 57-70.
2. Sawyers C (2004) Targeted cancer therapy. Nature 432: 294-297.
3. Giaccone G, Herbst R S, Manegold C, Scagliotti G, Rosell R, Miller V, Natale R B, Schiller J H, Von Pawel J, Pluzanska A, Gatzemeier U, Grous J, Ochs J S, Averbuch S D, Wolf M K, Rennie P, Fandi A and Johnson D H (2004) Gefitinib in combination with gemcitabine and cisplatin in advanced non-small-cell lung cancer: a phase III trial—INTACT 1. J. Clin. Oncol. 22: 777-784.
4. Herbst R S, Giaccone G, Schiller J H, Natale R B, Miller V, Manegold C, Scagliotti G, Rosell R, Oliff I, Reeves J A, Wolf M K, Krebs A D, Averbuch S D, Ochs J S, Grous J, Fandi A and Johnson D H (2004) Gefitinib in combination with paclitaxel and carboplatin in advanced non-small-cell lung cancer: a phase III trial—INTACT 2. J. Clin. Oncol. 22: 785-794.
5. Gatzemeier U, Pluzanska A, Szczesna A, Kaukel E, Roubec J, Brennscheidt U, De Rosa F, Mueller B and Von Pawel J. (2004) Results of a phase III trial of erlotinib (OSI-774) combined with cisplatin and gemcitabine (GC) chemotherapy in advanced non-small cell lung cancer. J. Clin. Oncol. 22: 7010.
6. Hecht J R, Trarbach T, Jaeger E, Hainsworth J, Wolff R, Lloyd K, Bodoky G, Laurent D and Jacques C. (2005) A randomized, double-blind, placebo-controlled, phase III study in patients (Pts) with metastatic adenocarcinoma of the colon or rectum receiving first line chemotherapy with oxaliplatin/5-fluorouracil/leucovorin and PTK787/ZK 222584 or placebo (CONFIRM-1). J. Clin. Oncol. 23: LBA3.
7. Buchner K (2000) The role of protein kinase C in the regulation of cell growth and in signalling to the cell nucleus. J. Cancer Res. Clin. Oncol. 126: 1-11.
8. Fabbro D, Buchdunger E, Wood J, Mestan J, Hofmann F, Ferrari S, Mett H, O'Reilly T and Meyer T (1999) Inhibitors of protein kinases: CGP 41251, a protein kinase inhibitor with potential as an anticancer agent. Pharmacol. Ther. 82: 293-301.
9. Tenzer A, Zingg D, Rocha S, Hemmings B, Fabbro D, Glanzmann C, Schubiger P A, Bodis S and Pruschy M (2001) The phosphatidylinositide 3′-kinase/Akt survival pathway is a target for the anticancer and radiosensitizing agent PKC412, an inhibitor of protein kinase C. Cancer Res. 61: 8203-8210.
10. Begemann M, Kashimawo S A, Heitjan D F, Schiff P B, Bruce J N and Weinstein I B (1998) Treatment of human glioblastoma cells with the staurosporine derivative CGP 41251 inhibits CDC2 and CDK2 kinase activity and increases radiation sensitivity. Anticancer Res. 18: 2275-2282.
11. Yoshida H, Kong Y-Y, Yoshida R, Elia A J, Hakem A, Hakem R, Penninger J M and Mak T W (1998) Apaf-1 Is Required for Mitochondrial Pathways of Apoptosis and Brain Development. Cell 94: 739-750.
12. Lindsten T, Ross A J, King A, Zong W-X, Rathmell J C, Shiels H A, Ulrich E, Waymire K G, Mahar P, Frauwirth K, Chen Y, Wei M, Eng V M, Adelman D M, Simon M C, Ma A, Golden J A, Evan G, Korsmeyer S J, MacGregor G R and Thompson C B (2000) The Combined Functions of Proapoptotic Bcl-2 Family Members Bak and Bax Are Essential for Normal Development of Multiple Tissues. Mol. Cell 6: 1389-1399.
13. Wei M C, Zong W-X, Cheng E H Y, Lindsten T, Panoutsakopoulou V, Ross A J, Roth K A, MacGregor G R, Thompson C B and Korsmeyer S J (2001) Proapoptotic BAX and BAK: A Requisite Gateway to Mitochondrial Dysfunction and Death. Science 292: 727-730.
14. Oltvai Z N, Milliman C L and Korsmeyer S J (1993) Bcl-2 heterodimerizes in vivo with a conserved homolog, Bax, that accelerates programmed cell death. Cell 74: 609-619 15. Chittenden T, Harrington E A, O'Connor R, Flemington C, Lutz R J, Evan G I and Guild B C (1995) Induction of apoptosis by the Bcl-2 homologue Bak. Nature 374: 733-736.
16. Jürgensmeier J M, Xie Z, Deveraux Q, Ellerby L, Bredesen D and Reed J C (1998) Bax directly induces release of cytochrome c from isolated mitochondria. Proc. Natl. Acad. Sci. USA 95: 4997-5002.
17. Finucane D M, Bossy-Wetzel E, Waterhouse N J, Cotter T G and Green D R (1999) Bax-induced Caspase Activation and Apoptosis via Cytochrome c Release from Mitochondria Is Inhibitable by Bcl-xL. J. Biol. Chem. 274: 2225-2233.
18. Chen L, Willis S N, Wei A, Smith B J, Fletcher J I, Hinds M G, Colman P M, Day C L, Adams J M and Huang D C S (2005) Differential Targeting of Prosurvival Bcl-2 Proteins by Their BH3-Only Ligands Allows Complementary Apoptotic Function. Mol. Cell 17: 393-403.
19. Kuwana T, Bouchier-Hayes L, Chipuk J E, Bonzon C, Sullivan B A, Green D R and Newmeyer D D (2005) BH3 Domains of BH3-Only Proteins Differentially Regulate Bax-Mediated Mitochondrial Membrane Permeabilization Both Directly and Indirectly. Mol. Cell 17: 525-535.
20. Vaux D L, Cory S and Adams J M (1988) Bcl-2 gene promotes haemopoietic cell survival and cooperates with c-myc to immortalize pre-B cells. Nature 335: 440-442.
21. Strasser A, Harris A W, Bath M L and Cory S (1990) Novel primitive lymphoid tumours induced in transgenic mice by cooperation between myc and bcl-2. Nature 348: 331-333.
22. Schmitt C A, Fridman J S, Yang M, Baranov E, Hoffman R M and Lowe S W (2002) Dissecting p53 tumor suppressor functions in vivo. Cancer Cell 1: 289-298.
23. Green D R, Evan G I (2002) A matter of life and death. Cancer Cell 1: 19-30.
24. Kaufmann S H, Vaux D L (2003) Alterations in the apoptotic machinery and their potential role in anticancer drug resistance. Oncogene 22: 7414-7430.
25. Johnstone R W, Ruefli A A and Lowe S W (2002) Apoptosis: A Link between Cancer Genetics and Chemotherapy. Cell 108: 153-164.
26. Huber C, Bobek N, Kuball J, Thaler S, Hoffarth S, Huber C, Theobald M and Schuler M (2005) Inhibitors of apoptosis confer resistance to tumour suppression by adoptively transplanted cytotoxic T lymphocytes in vitro and in vivo. Cell Death Diff. 12: 317-325.
27. Fukuoka M, Yano S, Giaccone G, Tamura T, Nakagawa K, douillard J Y, Nishiwaki Y, Vansteenkiste J, Kudoh S, Rischin D, Eek R, Horai T, Noda K, Takata I, Smit E, Averbuch S D, Macleod A, Feyereislova A, Dong R P and Baselga J (2003) Multi-institutional randomized phase II trial of gefitinib for previously treated patients with advanced non-small-cell lung cancer (The IDEAL 1 Trial). J. Clin. Oncol. 21: 2237-2246.
28. Shepherd F A, Pereira J, Ciuleanu T E, Tan E H, Hirsh V, Thongprasert S, Bezjak A, tu D, Santabarbara P, Seymour L and NCIC CTG. (2004) A randomized placebo-controlled trial of erlotinib in patients with advanced non-small cell lung cancer (NSCLC) following failure of 1st line or 2nd line chemotherapy. A National Cancer Institute of Canada Clinical Trials Group (NCIC CTG) trial. J. Clin. Oncol. 22: 7022.
29. Lynch T J, Bell D W, Sordella R, Gurubhagavatula S, Okimoto R A, Brannigan B W, Harris P L, Haserlat S M, Supko J G, Haluska F G, Louis D N, Christiani D C, Settleman J and Haber D A (2004) Activating mutations in the epidermal growth factor receptor underlying responsiveness of non-small-cell lung cancer to gefitinib. N. Engl. J. Med. 350: 2129-2139.
30. Paez J G, Jänne P A, Lee J C, Tracy S, Greulich H, Gabriel S, Herman P, Kaye F J, Lindeman N, Boggon T J, Naoki K, Saski H, Fuji Y, Eck M J, Sellers W R, Johnson B E and Meyerson M (2004) EGFR Mutations in Lung Cancer: Correlation with Clinical Response to Gefitinib Therapy. Science 304: 1497-1500.
31. Andrejauskas-Buchdunger E, Regenass U (1992) Differential inhibition of the epidermal growth factor-, platelet-derived growth factor-, and protein kinase C-mediated signal transduction pathways by the staurosporine derivative CGP 41251. Cancer Res. 52: 5353-5358.
32. Joseph B, Marchetti P, Formstecher P, Kroemer G, Lewensohn R and Zhivotovsky B (2002) Mitochondrial dysfunction is an essential step for killing of non-small cell lung carcinomas resistant to conventional treatment. Oncogene 21: 65-77.
33. Propper D J, McDonald A C, Man A, Thavasu P, Balkwill F, Braybrooke J P, Caponigro F, Graf P, Dutreix C, Blackie R, Kaye S B, Ganesan T S, Talbot D C, Harris A L and Twelves C (2001) Phase I and pharmacokinetic study of PKC412, an inhibitor of protein kinase C. J. Clin. Oncol. 19: 1485-1492.
34. Monnerat C, Henriksson R, Le Chevalier T, Novello S, Berthaud P, Faivre S and Raymond E (2004) Phase I study of PKC412 (N-benzoyl-staurosporine), a novel oral protein kinase C inhibitor, combined with gemcitabine and cisplatin in patients with non-small-cell lung cancer. Ann. Oncol. 15: 316-323.
35. Hemström T H, Joseph B, Schulte G, Lewensohn R and Zhivotovsky B (2005) PKC 412 sensitizes U1810 non-small cell lung cancer cells to DNA damage. Exp. Cell Res. 305: 200-213.
36. Walensky L D, Kung A L, Escher I, Malia T J, Barbuto S, Wright R D, Wagner G, Verdine G L and Korsmeyer S J (2004) Activation of Apoptosis in Vivo by a Hydrocarbon-Stapled BH3 Helix. Science 305: 1466-1470.
37. Oltersdorf T, Elmore S W, Shoemaker A R, Armstrong R C, Augeri D J, Belli B A, Bruncko M, Deckwerth T L, Dinges J, Hadjuk P J, Joseph M K, Kitada S, Korsmeyer S J, Kunzer A R, Letai A, Li C, Mitten M J, Nettesheim D G, Ng S, Nimmer P M, O'Connor J M, Oleksijew A, Petros A M, Reed J C, Shen W, Tahir S K, Thompson C B, Tomaselli K J, Wang B, Wendt M D, Zhang H, Fesik S W and Rosenberg S H (2005) An inhibitor of Bcl-2 family proteins induces regression of solid tumours. Nature 435: 677-681.
38. Schuler M, Maurer U, Goldstein J C, Breitenbücher F, Hoffarth S, Waterhouse N J and Green D R (2003) p53 triggers apoptosis in oncogene-expressing fibroblasts by the induction of Noxa and mitochondrial Bax translocation. Cell Death Diff. 10: 451-460.
39. Milosevic J, Hoffarth S, Huber C and Schuler M (2003) The DNA damage-induced decrease of Bcl-2 is secondary to the activation of apoptotic effector caspases. Oncogene 22: 6852-6856.
40. Reed J C, Pellecchia M. (2005) Apoptosis-based therapies for hematologic malignancies. Blood. 106: 408-418. Electronic publication 2005 Mar. 29.

1. A method of treating or preventing non-small cell lung cancer, the method comprising administering a staurosporine derivative selected from a compound of formula (II) or (III): 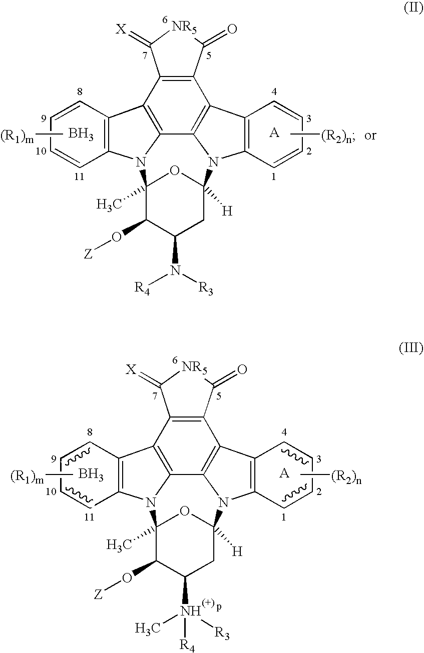
wherein the compound (III) is the partially hydrogenated derivative of compound (II); or staurosporine derivatives of formula (IV) or (V) or (VI) or (VII): 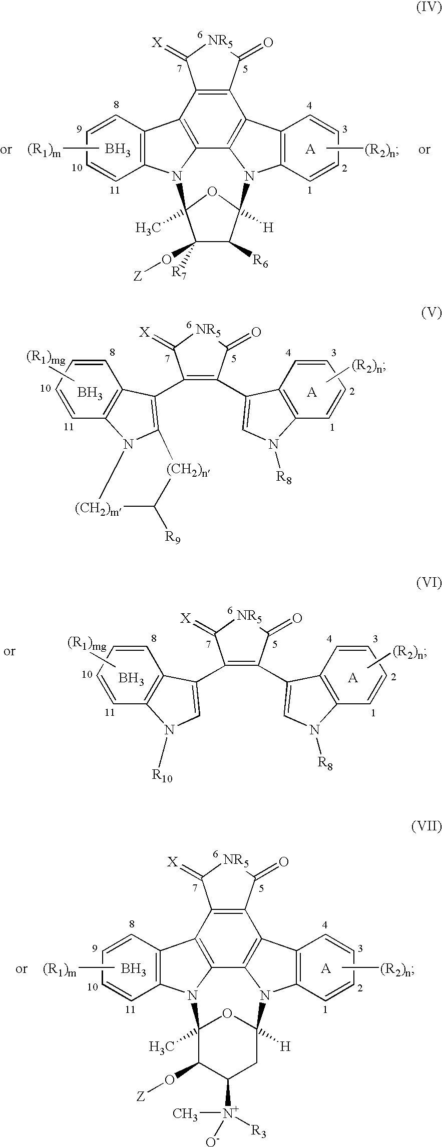
wherein R₁ and R₂, are, independently of one another, unsubstituted or substituted alkyl, hydrogen, halogen, hydroxyl, etherified or esterified hydroxyl, amino, mono- or disubstituted amino, cyano, nitro, mercapto, substituted mercapto, carboxy, esterified carboxy, carbamoyl, N-mono- or N,N-di-substituted carbamoyl, sulfo, substituted sulfonyl, aminosulfonyl or N-mono- or N,N-di-substituted aminosulfonyl;
n and m are, independently of one another, a number from and including 0 to and including 4;
n′ and m′ are, independently of one another, a number from and including 0 to and including 4;
R₃, R₄, R₈ and R₁₀ are, independently of one another, hydrogen, —O⁻, acyl with up to 30 carbon atoms, an aliphatic, carbocyclic, or carbocyclic-aliphatic radical with up to 29 carbon atoms in each case, a heterocyclic or heterocyclic-aliphatic radical with up to 20 carbon atoms in each case, and in each case up to 9 heteroatoms, an acyl with up to 30 carbon atoms, wherein R₄ may also be absent;
or if R₃ is acyl with up to 30 carbon atoms, R₄ is not an acyl;
p is 0 if R₄ is absent, or is 1 if R₃ and R₄ are both present and in each case are one of the aforementioned radicals;
R₅ is hydrogen, an aliphatic, carbocyclic, or carbocyclic-aliphatic radical with up to 29 carbon atoms in each case, or a heterocyclic or heterocyclic-aliphatic radical with up to 20 carbon atoms in each case, and in each case up to 9 heteroatoms, or acyl with up to 30 carbon atoms;
R₇, R₆ and R₉ are acyl or -(lower alkyl)-acyl, unsubstituted or substituted alkyl, hydrogen, halogen, hydroxyl, etherified or esterified hydroxyl, amino, mono- or disubstituted amino, cyano, nitro, mercapto, substituted mercapto, carboxy, carbonyl, carbonyldioxy, esterified carboxy, carbamoyl, N-mono- or N,N-di-substituted carbamoyl, sulfo, substituted sulfonyl, aminosulfonyl or N-mono- or N,N-di-substituted aminosulfonyl;
X stands for 2 hydrogen atoms; for 1 hydrogen atom and hydroxyl; for O; or for hydrogen and lower alkoxy;
Z stands for hydrogen or lower alkyl;
and either the two bonds characterised by wavy lines are absent in ring A and replaced by 4 hydrogen atoms, and the two wavy lines in ring B each, together with the respective parallel bond, signify a double bond;
or the two bonds characterised by wavy lines are absent in ring B and replaced by a total of 4 hydrogen atoms, and the two wavy lines in ring A each, together with the respective parallel bond, signify a double bond;
or both in ring A and in ring B all of the 4 wavy bonds are absent and are replaced by a total of 8 hydrogen atoms;
or a salt thereof, if at least one salt-forming group is present;
wherein the tyrosine kinase inhibitor treats or prevents non-small cell lung cancer.

2. The method according to claim 0, wherein non-small cell lung cancer is sensitive to cytotoxic anticancer drugs

3. The method according to claim 0, wherein the treatment further comprises administering a topoisomerase inhibitor.

4. The method according to claim 0, wherein the topoisomerase inhibitor is VP16.

5. The method according to claim 0 where the non-small cell lung cancer has resistance to cytotoxic anticancer drugs.

6. The method according to claim 0, wherein the treatment further comprises administering a modulator of BAK activity.

7. The method according to claim 0, wherein the modulator is an activator of BAK activity.

8. The method according to claim 0, wherein the treatment further comprises administering a composition that enhances mitochondrial outer membrane permeabilization.

9. The method according to claim 0 wherein the non-small cell lung cancer is associated with a FLT-3 mutation.

10. The method according to claim 0, wherein the tyrosine kinase inhibitor is a compound of formula (I): 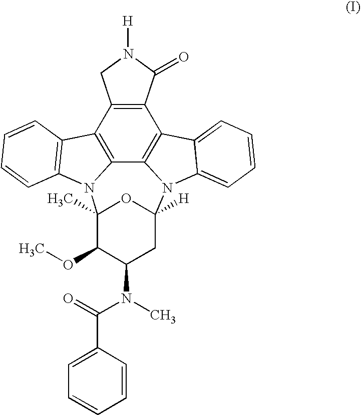
or pharmaceutically acceptable salts thereof.

11. (canceled)

12. (canceled)

13. A method for treating mammals suffering from non-small cell lung cancer comprising administering to a mammal in need of such treatment a tyrosine kinase-inhibiting amount of a compound of formula (I): 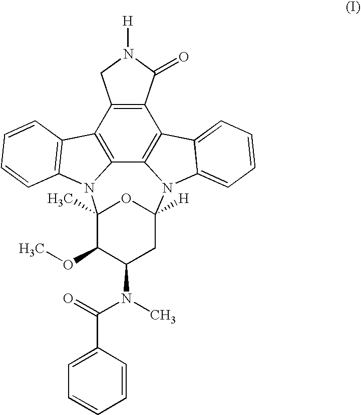
or pharmaceutically acceptable salts thereof.

14. A method according to claim 0, therein the mammal is a human.

15. (canceled)

16. A method of treating non-small cell lung cancer in a mammal that comprises treating the mammal in need of such treatment simultaneously, concurrently, separately or sequentially with pharmaceutically effective amounts of (a) a FLT-3 inhibitor, or a pharmaceutically acceptable salt or a prodrug thereof, and (b) a modulator of BAK activity, or a pharmaceutically acceptable salt or a prodrug thereof.

17. (canceled)

18. (canceled)

19. A method according to claim 16, wherein the FLT-3 inhibitor is N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-Im]pyrrolo[3,4-j][1,7]benzodiazonin-11-yl]-N-methylbenzamide of the formula (I): 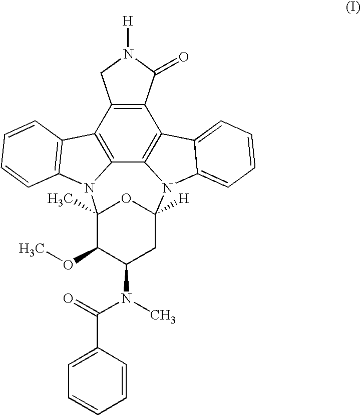
or a salt thereof.

20. A method of claim 19, wherein the salt is a pharmaceutically acceptable salt.

21. A method of inducing drug sensitivity in a drug-resistant cancer cell, the method comprising inducing the apoptotic signal transduction pathway in the cancer cell.

22. The method of claim 21, wherein the method comprises administering at least one activator of BAK activity.

23. The method of claim 21, wherein the method comprises administering at least one inhibitor of Bcl-1/Bcl-XL activity.

24. The method of claim 21, wherein the sensitivity induced in the cancer cell is to a drug comprising a staurosporine derivative.

25. A method of treating drug-resistant cancer cells, the method comprising administering to a cancer cell an inducer of apoptosis and a staurosporine derivative.
